(19) United States
(12) Patent Application Publication
(10) Pub. No.: US 2023/0286893 A1
(43) Pub. Date: Sep. 14, 2023

(54) VANILLYLIDENECHLOROACETONE AND ITS USE THEREOF IN SYNTHESIS OF CALEBIN-A

(71) Applicants: Muhammed Majeed, Bangalore (IN); Kalyanam Nagabhushanam, East Windsor, NJ (US); Nagarajan Muthukaman, Bangalore (IN); Paradesi Naidu Pentakota, Bangalore (IN)

(72) Inventors: Muhammed Majeed, Bangalore (IN); Kalyanam Nagabhushanam, East Windsor, NJ (US); Nagarajan Muthukaman, Bangalore (IN); Paradesi Naidu Pentakota, Bangalore (IN)

(21) Appl. No.: 18/116,987

(22) Filed: Mar. 3, 2023

Related U.S. Application Data
(60) Provisional application No. 63/318,047, filed on Mar. 9, 2022.

(51) Int. Cl. C07C 49/255; C07C 45/69; C07C 67/10

(52) U.S. Cl. CPC C07C 49/255; C07C 45/69; C07C 67/10

(57) ABSTRACT

The present invention discloses a compound, Vanillylidenechloroacetone (VCA)—1-Chloro-4-(4′-hydroxy-3′-methoxy-phenyl)-but-3-en-2-one, and the process of preparing the same. The invention also discloses a process for synthesis of Calebin-A from Vanillylidenechloroacetone.

### CROSS-REFERENCE TO RELATED APPLICATIONS

This application is a PCT filing claiming priority from U.S. provisional application No. 63/318,047 filed on 9 Mar. 2022, the contents of which are incorporated herein by reference.

### FIELD OF INVENTION

The present invention in general relates to a novel compound, Vanillylidenechloroacetone (VCA), 1-Chloro-4-(4′-hydroxy-3′-methoxy-phenyl)-but-3-en-2-one. More specifically, the invention relates to a process to prepare VCA and use of the same in the synthesis of Calebin-A.

### DESCRIPTION OF PRIOR ART

Calebin-A, also known as Feruloylmethyl ferulate, is a novel curcuminoid related structure obtained from rhizomes of Curcuma longa, of Zingiberaceae family. Calebin-A is naturally present in the rhizomes of Curcuma longa in trace amounts. It was first isolated and identified by Darrick S. H. L. Kim and So-Young Park (J. Nat. Prod., 2002, 65, 1227-1231) from Curcuma longa in 2001. Its analogs were synthesized by Kim, D. S. H. L. & Kim, J. Y. in 2001 (Bioorg. Med. Chem. Lett., 2001, 11, 2541-2543). Calebin-A has been reported to elicit various biological functions, some of which are mentioned in the following prior art documents:
1. Calebin A, a novel component of turmeric, suppresses NF-κB regulated cell survival and inflammatory gene products leading to inhibition of cell growth and chemosensitization, Tyagi et. al., Phytomedicine. 2017 Oct. 15; 34: 171-181.
2. Calebin-A induces cell cycle arrest in human colon cancer cells and xenografts in nude mice, Liou et. al., Journal of Functional Foods, Volume 26, October 2016, Pages 781-791
3. Majeed et al., Anti-obesity potential of Calebin A, U.S. Pat. No. 9,328,330.
4. Majeed et. al., Method for the treatment of hypercholesterolemia, U.S. Pat. No. 9,668,999.
5. Majeed et. al., Calebin A for hepatic steatosis, U.S. Pat. No. 9,737,502.
6. Majeed et. al., Composition and method for the protection of articular cartilage, U.S. Pat. No. 9,220,703.
7. Majeed et. al., Calebin A for osteoporosis, U.S. Pat. No. 9,539,232

There are many commercial processes available for the preparation of Calebin-A and its pharmaceutically acceptable salts. Calebin-A and its derivatives were isolated and synthesised by Yong et. al., (Chem. Pharm. Bull. 55 (6) 940-943 (2007)) and Majeed et. al., (Georg Thieme Verlag Stuttgart⋅New York—SynOpen 2017, 1, 125-128). Majeed et. al. also disclosed a process for the synthesis of Calebin-A and its biologically active analogs (U.S. Pat. No. 9,365,486).

However, the above processes either involves multi-steps or commercially not viable. Hence there is need to develop a commercially viable, safe, simple and scalable process for the large-scale production of Calebin-A and its analogs.

The present invention fulfils the aforesaid objectives by disclosing a simple, scalable, industrially advantageous and cost-effective process for the synthesis of Calebin-A. The invention also discloses a novel compound, Vanillylidenechloroacetone (VCA)—1-Chloro-4-(4-hydroxy-3-methoxy-phenyl)-but-3-en-2-one, used in the synthesis of Calebin A.

### SUMMARY OF THE INVENTION

In the most preferred embodiment of the invention discloses a compound, Vanillylidenechloroacetone (VCA)—1-Chloro-4-(4′-hydroxy-3′-methoxy-phenyl)-but-3-en-2-one, as represented by STR#1 and the process of preparing the same.

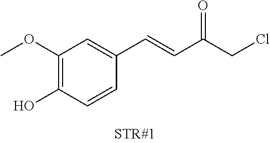

In yet another preferred embodiment of the invention, the invention discloses a process to prepare Calebin-A from VCA.

Other features and advantages of the present invention will become apparent from the following more detailed description.

### DETAILED DESCRIPTION OF THE MOST PREFERRED EMBODIMENT

In the most preferred embodiment of the invention discloses a compound, Vanillylidenechloroacetone (VCA)—1-Chloro-4-(4′-hydroxy-3′-methoxy-phenyl)-but-3-en-2-one VCA, as represented by STR#1.

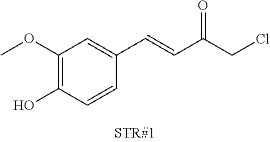

In another most preferred embodiment of the invention discloses a process to prepare VCA comprising steps of:
a) Reacting 1,3-dichloroacetone with R₃P in methanol at room temperature, gradually heating to above room temperature for a time period, allowing it to go for completion, and cooling to room temperature to generate a reaction mixture;
b) Quenching the mixture from step a) with an aqueous base and maintaining it at room temperature for 20-24 hrs;
c) Processing the reaction mixture from step b) to isolate the resulting ylid as represented by STR#2; and

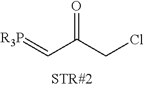

d) Refluxing the ylid from step c) with Vanillin in a solvent for 15 hours and isolating the compound VCA, as represented by STR#1,

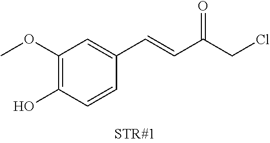

wherein VCA is isolated with a yield of not less than 40%.

In related embodiments of the invention, R in R₃ (STR#2) is preferably alkyl, or more preferably aryl group, wherein alkyl group is C1-C4 linear or substituted, aryl group is either phenyl or substituted phenyl. In a preferred embodiment R is a phenyl group and in this preferred embodiment R₃P is triphenylphosphine. In another related embodiment of the invention, above room temperature ranges from 35° C. to 70° C., and the time period ranges from 20 to 24 hours. In another preferred embodiment, the yield of VCA is in the range 40% to 60%. In another related embodiment of the invention, the aqueous base is selected from the group consisting of aqueous sodium carbonate, sodium hydroxide, potassium hydroxide, lithium hydroxide, calcium hydroxide, or carbonates such as sodium carbonate, potassium carbonate, caesium carbonate, calcium carbonate, magnesium carbonate, sodium bicarbonate, potassium bicarbonate, or sodium methoxide, or potassium methoxide, or triethylamine, or tributylamine, or N-methylmorpholine, or N,N-diisopropylethylamine, or N-methylpyrrolidine, or pyridine, or collidine 4-(N,N-dimethylamino)pyridine, or morpholine, or imidazole, or 2-methylimidazole, or 4-methylimidazole. Any reference to processing in this embodiment (step c) refers to filtering, washing with methanol, and purification. The solvent in this embodiment includes protic or aprotic solvents, selected from the group consisting of water, methanol, ethanol, isopropanol, acetone, methyl ethyl ketone, diethyl ketone, methyl isopropyl ketone, methyl isobutyl ketone, diethyl ether, diisopropyl ether, tert-butyl methyl ether, dibutyl ether, tetrahydrofuran, 1,2-dimethoxyethane, 2-methoxyethanol, 2-ethoxy ethanol, anisole, 1,4-dioxane, toluene, xylene, chlorobenzene, tetralin, chloroform, dichloromethane, DMF, acetic acid or mixtures thereof.

The choice of base and solvent are within the scope of those skilled in the art.

In yet another preferred embodiment of the invention, a process to prepare Calebin-A from VCA, comprising steps of:

Reacting 1-Chloro-4-(4′-hydroxy-3′-methoxy-phenyl)-but-3-en-2-one with ferulic acid at room temperature in a solvent in the presence of a base;

Isolating Calebin-A as represented by STR#3, from step a) with an yield of at least 80%.

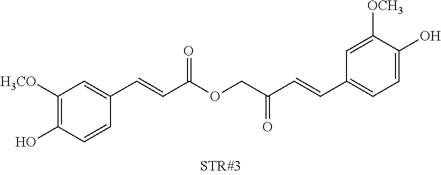 [SMILES: COc1cc(/C=C/C(=O)COC(=O)/C=C/c2ccc(O)c(OC)c2)ccc1O]

In a related embodiment of the invention, the solvent is selected from the group consisting of protic or aprotic solvents, selected from the group consisting of water, methanol, ethanol, isopropanol, acetone, methyl ethyl ketone, diethyl ketone, methyl isopropyl ketone, methyl isobutyl ketone, diethyl ether, diisopropyl ether, tert-butyl methyl ether, dibutyl ether, tetrahydrofuran, 1,2-dimethoxyethane, 2-methoxyethanol, 2-ethoxy ethanol, anisole, 1,4-dioxane, toluene, xylene, chlorobenzene, tetralin, chloroform, dichloromethane, DMF, acetic acid or mixtures thereof. In another embodiment, the base is preferably Alkali metal or alkaline earth metal hydroxides such as sodium hydroxide, potassium hydroxide, lithium hydroxide, calcium hydroxide; carbonates such as sodium carbonate, potassium carbonate, caesium carbonate, calcium carbonate, magnesium carbonate, sodium bicarbonate, potassium bicarbonate, alkoxides such as sodium methoxide, potassium methoxide; organic bases, such as for example, triethylamine, tributylamine, N-methylmorpholine, N,N-diisopropylethylamine, N-methylpyrrolidine, pyridine, collidine 4-(N,N-dimethylamino)pyridine, morpholine, imidazole, 2-methylimidazole, 4-methylimidazole.

In another aspect of the invention, the yield is in the range 80% to 95%.

The choice of base and solvent are within the scope of those skilled in the art.

### EXAMPLES

### Example-1: Preparation of 1-Chloro-3-(triphenyl-λ5-phosphanylidene)-propan-2-one

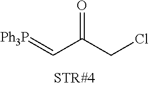

### Scheme:

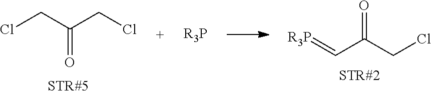

To a solution of 1,3-dichloroacetone (STR#5) (5 g, 0.0393 mol) in methanol (30.0 mL), charged triphenylphosphine (R=phenyl in the scheme, 8.26 g, 0.0314 mol) and stirred for 30 min at room temperature. The reaction mass was then heated to 60° C., maintained for 20 h and check the TLC. After the completion of reaction, cooled the reaction to room temperature, quench the reaction mass with 10% of aqueous sodium carbonate solution (15 ml) and maintain reaction for 12 h at room temperature. Filter the reaction mass and washed with aqueous methanol to afford 1-Chloro-3-(triphenyl-λ5-phosphanylidene)-propan-2-one (STR#4, Wittig ylid) (4.5 g, 98.48% HPLC) as pale white solid. Melting Point: 154-156° C., [M+H]⁺=353.
¹H NMR (300 MHz, DMSO-d₆): δ 4.11 (s, 2H), 7.45-7.75 (m, 15H).
¹³C NMR (100 MHz, DMSO-d₆): 47.68, 129.53, 133.05, 183.86.

### Example-2: Preparation of VCA, STR#1

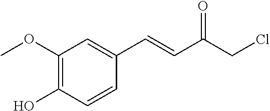

### Scheme:

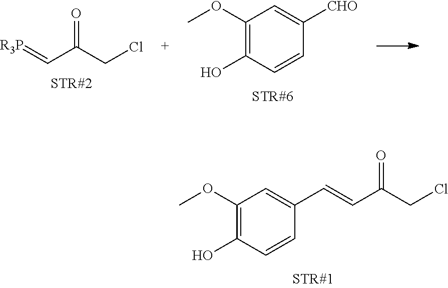

To a solution of vanillin (5 g, 0.0257 mol) in toluene (15.0 mL), charged 1-chloro-3-(triphenyl-λ5-phosphanylidene)-propan-2-one (STR#2) (6.54 g, 0.0515 mol) and stirred for 30 min at room temperature. The reaction mass was heated to 100-110° C., maintained for 15 h and cooled to room temperature. Add water (15 ml) and ethyl acetate (25 ml) to the above reaction mass and stirred for 30 mins. The organic layer (ethyl acetate+toluene) was washed with sodium bicarbonate solution (20 ml), followed by sodium chloride solution. Distilled the total ethyl acetate and isolated the solid in toluene and dried the compound under reduced pressure (vacuum) to yield 1-chloro-4-(4′-hydroxy-3′-methoxy-phenyl)-but-3-en-2-one (STR#1) as white solid. Yield: 3.5 g; 47% (Mp: 55-65° C.; [M+H]⁺=227)
1H NMR (300 MHz, DMSO-d₆): δ 4.07 (s, 3H), 6.03 (s, 2H), 6.85-6.80 (d, J=15.0 Hz, 1H) 6.96-6.93 (d, J=9.0 Hz, 1H), 7.07-7.03 (d, J=7.5 Hz, 1H), 7.44-7.42 (d,1H), 7.67-7.62 (d, J=15.0 Hz, 1H).
¹³C NMR (100 MHz, DMSO-d₆): 56.08, 115.76, 116.08, 120.40, 124.32, 125.93, 146.70, 145.15, 148.40, 150.35, 191.43.

### Process to Prepare Cale (STR#3) from VCA (STR#1)

### Scheme:

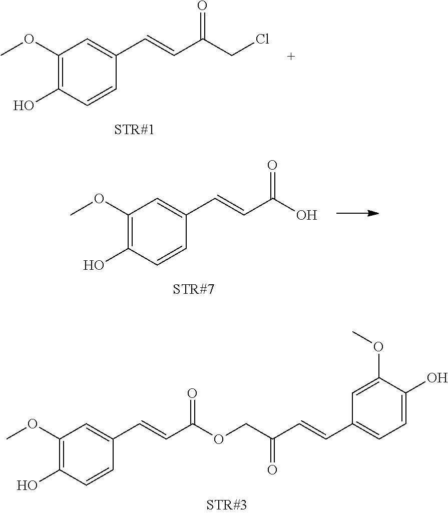 [SMILES: COc1cc(/C=C/C(=O)CCl)ccc1O ; COc1cc(/C=C/C(=O)O)ccc1O ; COc1cc(/C=C/C(=O)COC(=O)/C=C/c2ccc(O)c(OC)c2)ccc1O]

To a solution of 1-Chloro-4-(4′-hydroxy-3′-methoxy-phenyl)-but-3-en-2-one (VCA, (STR#1)) (5 g, 0.0221 mol) in N,N-dimethyl formamide (15.0 mL), charged Ferulic Acid (STR#7) (4.3 g, 0.1221mol) & sodium bicarbonate (2.1 g, 0.02428) and stirred for 30 min at room temperature. The reaction mass was then heated to 50-55° C., maintained for 20 h and cooled to room temperature. After the completion of reaction, water (15 ml) and ethyl acetate (25 ml) were added to the above reaction mass. The separated organic layer was washed with sodium bicarbonate (20 ml) and brine solution. The ethyl acetate layer was distilled-off and diluted with toluene to afford solid. The resultant solid was filtered and dried at 80-85° C. for 15 h under reduced pressure to yield Calebin-A, 7 g, 81%.
¹H NMR (Acetone,-d₆, 300 MHz): δ 3.902 (s, 3H), 3.929 (s, 3H), 5.106 (s, 2H), 6.518 (d, J=15.9 Hz, 1H), 6.856 (d, J=16.2 Hz, 1H), 6.886 (d, J=8.4 Hz, 1H), 6.892 (d, J=8.4 Hz, 1H), 7.180 (dd, J=8.4 Hz, 1.8 Hz, 1H), 7.214 (dd, J=8.4 Hz, 1.8 Hz, 1H), 7.375 (d, J=1.8 Hz, 1H), 7.386 (d, J=1.8 Hz, 1H), 7.666 (d, J=16.2 Hz. 1H), 7.671 (d, J=15.9 Hz, 1H), 8.246 (s, 1H), 8.309 (s, 1H).
¹³C NMR (Acetone-d₆, 75 MHz): δ 56.267, 56.296, 67.927, 111.290, 111.524, 116.077, 116.201, 120.322, 124.165, 124.494, 127.349, 127.408, 1.44.389, 146.505, 148.752, 150.209, 150.501, 166.898, 192.963.

While the invention has been described with reference to a preferred embodiment, it is to be clearly understood by those skilled in the art that the invention is not limited thereto. Rather, the scope of the invention is to be interpreted only in conjunction with the appended claims.

1. A compound, 1-Chloro-4-(4′-hydroxy-3′-methoxy-phenyl)-but-3-en-2-one (VCA), as represented by STR#1

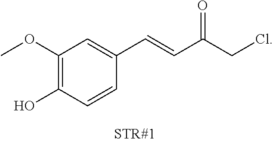

2. The compound as in claim 1, wherein the said compound is prepared using a process comprising steps of:
a) Reacting 1,3-dichloroacetone with R₃P in methanol at room temperature, gradually heating to above room temperature for a time period, allowing it to go for completion, and cooling to room temperature to generate a reaction mixture;
b) Quenching the reaction mixture from step a) with an aqueous base and maintaining it at room temperature for 20-24 hrs;
c) Processing the mixture from step b) to isolate the ylid as represented by STR#2; and 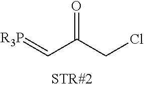
d) Refluxing the ylid from step c) with Vanillin in a solvent for 15 hours and isolating the compound VCA as represented by STR#1, 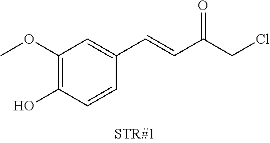
wherein VCA is isolated with an yield of not less than least 40%.

3. The process as in claim 2, wherein R in R₃P is selected from the group consisting of alkyl and aryl, wherein alkyl group is C1-C4 linear or substituted, aryl group is either phenyl or substituted phenyl.

4. The process as in claim 2, wherein the R in R₃P is phenyl.

5. The process as in claim 4, wherein R₃P is triphenylphosphine.

6. The process as in claim 2, wherein above room temperature ranges from 35° C. to 70° C.

7. The process as in claim 2, wherein the time period ranges from 20 hours to 24 hours.

8. The process as in claim 2, wherein the aqueous base is selected from the group consisting of sodium hydroxide, potassium hydroxide, lithium hydroxide, calcium hydroxide; sodium carbonate, potassium carbonate, caesium carbonate, calcium carbonate, magnesium carbonate, sodium bicarbonate, potassium bicarbonate, sodium methoxide, potassium methoxide, triethylamine, tributylamine, N-methylmorpholine, N,N-diisopropylethylamine, N-methylpyrrolidine, pyridine, collidine 4-(N,N-dimethylamino)pyridine, morpholine, imidazole, 2-methylimidazole, and 4-methylimidazole.

9. The process as in claim 2, wherein the processing in step c) involves filtering, washing with methanol, and purification.

10. The process as in claim 2, wherein the solvent is selected from the group consisting of methanol, ethanol, isopropanol, acetone, methyl ethyl ketone, diethyl ketone, methyl isopropyl ketone, methyl isobutyl ketone, ether solvents, diethyl ether, diisopropyl ether, tert-butyl methyl ether, dibutyl ether, tetrahydrofuran, 1,2-dimethoxyethane, 2-methoxyethanol, 2-ethoxy ethanol, anisole, 1,4-dioxane, toluene, xylene, chlorobenzene, tetralin, chloroform, dichloromethane, water, or mixtures thereof.

11. A process to prepare Calebin-A as represented by STR#3 from VCA, comprising steps of:
a) Reacting VCA with ferulic acid at room temperature in a solvent in the presence of a base;
b) Isolating Calebin-A from step a) with a yield of at least 80%

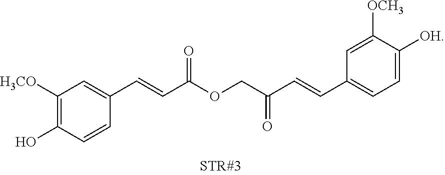 [SMILES: COc1cc(/C=C/C(=O)COC(=O)/C=C/c2ccc(O)c(OC)c2)ccc1O]

12. The process as in claim 11, wherein the solvent is selected from the group consisting of methanol, ethanol, isopropanol, acetone, methyl ethyl ketone, diethyl ketone, methyl isopropyl ketone, methyl isobutyl ketone, ether solvents, diethyl ether, diisopropyl ether, tert-butyl methyl ether, dibutyl ether, tetrahydrofuran, 1,2-dimethoxyethane, 2-methoxyethanol, 2-ethoxy ethanol, anisole, 1,4-dioxane, toluene, xylene, chlorobenzene, tetralin, chloroform, dichloromethane, water, or mixtures thereof.

13. The process as in claim 11, wherein the base is selected from the group consisting sodium hydroxide, potassium hydroxide, lithium hydroxide, calcium hydroxide; sodium carbonate, potassium carbonate, caesium carbonate, calcium carbonate, magnesium carbonate, sodium bicarbonate, potassium bicarbonate, sodium methoxide, potassium methoxide, triethylamine, tributylamine, N-methylmorpholine, N,N-diisopropylethylamine, N-methylpyrrolidine, pyridine, collidine 4-(N,N-dimethylamino)pyridine, morpholine, imidazole, 2-methylimidazole, and 4-methylimidazole.
